(19) United States
(12) Patent Application Publication
(10) Pub. No.: US 2022/0233546 A1
(43) Pub. Date: Jul. 28, 2022

(54) Neurologist Formulated Nocturnal Nootropic Founded on a Novel Theory of Brain Aging

(71) Applicant: Farah Fourcand, Metuchen, NJ (US)

(72) Inventor: Farah Fourcand, Metuchen, NJ (US)

(21) Appl. No.: 17/667,519

(22) Filed: Feb. 8, 2022

(51) Int. Cl. A61K 31/519; A61K 33/06; A61K 31/714; A61K 31/7024; A61K 31/12; A61K 31/352

(52) U.S. Cl. CPC A61K 31/519; A61K 33/06; A61K 31/352; A61K 31/7024; A61K 31/12; A61K 31/714

(57) ABSTRACT

Twelve evidence-based hypotheses form the theory of brain aging, herein referred to as Brain Theory. Herein proposed are bioactive, neuro-available substances that, in coaction, work duly to mitigate specific molecular mechanisms of brain aging and enhance cognitive function based on a respective Brain Theory hypothesis.

Brain Theory hypotheses are the following: (1) Neurological Reserve, (2) Reductive-Oxidative Stress, (3) Caloric Restriction Anti-Inflammation, (4) Homocysteine Metabolism, (5) Neurotransmitter Neuroplasticity, (6) Telomere Mortality, (7) Immunosenescence, (8) Proteinopathy, (9) Glymphatic Dysfunction, (10) Circadian Clock Epigenetics, (11) Calcium-Dependent Synaptic Plasticity, and the (12) Gut-Brain-Axis.

BrainTheory™ N^{o} 12 is a nocturnal dietary supplement formulated with evidence-based bioactive, neuro-available substances that modulate specific mechanisms of brain aging based on Brain Theory hypotheses. Substances in order of representative hypotheses are the following: (1) R-Alpha Lipoic Acid, (2) Crocetin, (3) Curcumin, (4) Methylcobalamin or Choline, (5) 5-Methyltetrahydrofolate or Eucommia ulmoides Oliver, (6) Trans-Pterostilbene, (7) Cholecalciferol or Omega-3 Fatty Acid Compound, (8) Apigenin, (9) Luteolin, (10) Melatonin, (11) Magnesium L-Threonate, and (12) Apple Pectin Prebiotic. Substances additionally have evidence-based cognitive enhancement properties akin to those of a nootropic agent.

A dietary supplement formulation duly purposed to modulate healthspan-related biological brain aging and demonstrate nootropic effects is not previously described.

### INTRODUCTION

Rational scientific explanations, or hypotheses, for maladaptive brain aging are well studied¹. However, a trusted scientific explanation, or theory, on the mechanisms of brain aging is not well-established and currently under robust investigation¹. Brain Theory is a proposed theory of brain aging developed by a physician-scientist neurologist and founded on high-quality level evidence, including but not limited to randomized clinical trials and critical appraisal of the scientific literature. A dietary supplement duly purposed to modulate maladaptive brain aging based on an evidence-based theory of brain aging as well as provide nootropic benefits, herein referred to as BrainTheory™ N^{o} 12, does not currently exist.

Modulation of Biological Brain Aging

Brain Theory is founded on the principle that maladaptive brain aging is a manifestation of biological age (healthspan) as opposed to chronological age (lifespan)². Early in life, molecular pathways controlled by genetic and epigenetic factors determine biological aging². The heterogeneous accumulation of mutations and coding errors during a lifespan determines if the aging process is adaptive or maladaptive². Bioactive, neuro-available substances in BrainTheory™ N^{o} 12 are proposed to modulate molecular aging pathways that affect biological brain aging.

Nootropic Effects

The proposed nootropic effects of BrainTheory™ N^{o} 12 are associated with the cognitive enhancement properties of each substance. Review of high-quality level evidence including but not limited to randomized controlled trials and critical appraisal of the scientific literature validates proposed claims of cognitive enhancement, bioactivity, neuro-availability, dose-dependence, nocturnal administration, safety, and tolerability.

### PROPOSAL

Nocturnal Biological Brain Aging Processes

Biological brain aging processes are constant and dynamic. However, there are sleep-dependent functions vital to preservation of brain function including but not limited to clearance of pathological brain aging biomarkers³ and learning and memory consolidation⁴. Moreover, neurodegenerative processes and dementia syndromes are preceded by clinical and/or subclinical disorganized sleeping patterns as well as impaired degradation and clearance of pathological proteins and toxic waste products⁵. Therefore, nocturnal administration of BrainTheory™ N^{o} 12 (1-3 hours prior to onset of sleep) is recommended for peak performance.

R-Alpha-Lipoic Acid and the Neurological Reserve Hypothesis

Modulation of Biological Brain Aging to Enhance Healthspan

The Neurological Reserve Hypothesis is also known as the Network Control Hypothesis^{6, 7}. Brain size (brain reserve) and the brain's ability to grow, evolve, and adapt (cognitive reserve) decreases during the normal brain aging processe^{6, 7, 8}. However, there is significant heterogeneity in the rate of decline of neurological reservese.^{6, 7, 8}. This is due to marked between-persons differences in the molecular resilience of the brain's neural network^{6, 7, 8}. Neurons with effective intercommunication have superior connectivity such that the ‘connectome’ is optimally activated and preserved^{6, 7, 8}.

The substance in BrainTheory™ N^{o} 12 that preserves brain and cognitive reserve is (R)-Alpha Lipoic Acid. The R-enantiomer is the bioactive and neuro-available form of Alpha Lipoic Acid, which is endogenous and produced in neuronal mitochondria⁹. (R)-Alpha Lipoic Acid is clinically shown to increase brain and cognitive reserve⁹. A randomized, controlled, double-blind human clinical trial showed that bioactive (R)-Alpha Lipoic Acid reduces yearly rate of brain atrophy in young persons with multiple sclerosis by nearly 70% with a good safety and tolerability profile⁹. Multiple sclerosis is a potential model of neurological reserve given that brain atrophy is part of the natural history and pathophysiology of this pathological disease process⁹.

Nootropic and Systemic Effects

Human studies demonstrate that bioactive (R)-Alpha Lipoic Acid is effective in alleviating symptoms of peripheral neuropathy¹⁰ and improves glucose and fat metabolism for weight control¹¹. Bioactive (R)-Alpha Lipoic Acid is superior to glutathione and independently enhances the systemic functions of glutathione, vitamins C, and vitamin E¹².

Crocetin and the Reductive-Oxidative Stress Hypothesis

Modulation of Biological Brain Aging to Enhance Healthspan

The Reductive-Oxidative Stress Hypothesis posits that neurons shift from a stable reduced state to an unstable oxidized state in normal brain aging¹³ whereby reduction and oxidation are inverses, the latter of which is characterized by the loss of electrons, loss of hydrogen, gain of oxygen, and increase in oxidation number.

In maladaptive brain aging, a majority of neurons shift from a stable, reduced state to an unstable, oxidized state that propagates the accumulation of regulatory errors and inhibits the mitigation of salvage pathways¹³. The sequelae of this maladaptive process is decreased capacity to maintain optimal cognitive function with age. This is also the rationale behind the use of antioxidants and free radical scavengers in nutritional and dietary supplementation¹³.

The substance in BrainTheory™ N^{o} 12 that mitigates redox stress is Crocetin. Crocetin is a bioactive, neuro-available component of saffron that has superior stability and blood-brain barrier penetrance compared to other saffron derivatives, including saffranal and crotin¹⁴. A randomized, controlled, double-blind human clinical trial demonstrated that bioactive saffron has similar effects to donepezil, a prescription medication for dementia, in mild to moderate Alzheimer's disease in under 6 months. The Alzheimer's Disease Assessment Scale-Cognitive Subscale that assesses word and procedural recall, naming, commands, construction and ideational praxis, orientation, word recognition, language, and comprehension was used for evaluation¹⁵.

Nootropic and Systemic Effects

Bioactive saffron is also superior to vitamin E for protection of the cell wall with less risk of toxicity. Cell wall stability determines the pathway of rate-limiting steps in brain aging¹⁶. Bioactive saffron regulates subclinical neurodegenerative processes that predate clinical manifestations of mild cognitive impairment^{17, 18}. Human studies demonstrate that bioactive saffron has a similar effect to methylphenidate in attention deficit disorder¹⁹, contains antidepressant and analgesic properties comparable to fluoxetine²⁰, and reduces opioid-related memory loss and opioid withdrawal symptoms²⁰. Laboratory models demonstrate that bioactive saffron has anti-epileptic properties and clinical efficacy in mitigating neuronal damage in stroke, traumatic brain injury, and spinal cord injury²⁰.

Curcumin and the Caloric Restriction Anti-Inflammation Hypothesis

Modulation of Biological Brain Aging to Enhance Healthspan

Akin to localized macroscopic inflammation from physical injury, molecular inflammation occurs systemically on a microscopic level²¹. In a subset of individuals, inflammation occurs at an accelerated rate with a fulminant course akin to a cytokine storm²¹. In brain aging, activation of specific neuron subtypes, namely astrocytes and microglia, precipitate neuroinflammation²¹.

The sequelae of this maladaptive process is decreased capacity to maintain cognitive function with age and accelerated neurodegenerative processes²¹. The scientific literature suggests that neuroinflammation can be mitigated in a multitude of ways²¹. However, caloric restriction has the strongest evidence that demonstrates attenuation of pro-inflammatory astrocytic and microglial processes²¹. Given the practical limitations of caloric restriction, mimickers of caloric restriction are well studied and have similar mechanisms of action in regulating inflammatory pathways²¹.

The substance in BrainTheory™ N^{o} 12 that regulates neuroinflammation is Curcumin. Curcumin is a bioactive and neuro-available component in turmeric that mimics caloric restriction in the brain and systemically²². A randomized, controlled, double-blind human clinical trial demonstrated that Curcumin improves working memory and attention in adults without dementia²². Tests for verbal memory (Buschke Selective Reminding Test), visual memory (Brief Visual Memory Test-Revised), and attention (Trail Making A) domains were used for evaluation²². Persons who underwent positron emission tomography to detect post-interventional changes in pathological proteins, including amyloid and tau, demonstrated a significant decrease in pathological proteins in brain regions including the amygdala²².

Nootropic and System Effects

Human studies demonstrate that Curcumin improves mood, anxiety, and fatigue in healthy adults²³. Curcumin plays a preventative role in Alzheimer's and other neurodegenerative diseases^{23, 24}. In athletes and non-athletes, Curcumin decreases biomarkers of stress and muscle damage²⁵. Curcumin has anti-obesity properties that limit fat cell growth in metabolic syndrome as well as improves hyperglycemia, insulin resistance, hypertension, hyperlipidemia, and arthritis^{26, 27}.

Methylcobalamin and Choline in the Homocysteine Metabolism Hypothesis

Modulation of Biological Brain Aging to Enhance Healthspan

Homocysteine is an amino acid that requires post-production modification mainly via the transfer of a methyl group²⁸. High levels of homocysteine lead to dysfunctional transfer of methyl groups²⁸. The main sequelae of this maladaptive process is hypermethylation of DNA and proteins that accelerate brain aging²⁸. An international consensus statement based on evidence from meta-analyses of human studies concluded that high levels of homocysteine are associated with age-associated neurodegenerative diseases including Parkinson's and Alzheimer's diseases, accelerated brain atrophy, chronic brain hypoxia, and ischemic stroke²⁹⁻³⁴.

The substance in BrainTheory™ N^{o} 12 that regulates homocysteine methylation is Methylcobalamin³⁵. Methylcobalamin is the bioactive and neuro-available form of vitamin B12 that has superior bioavailability compared to cyanocobalamin³⁵. A meta-analysis of 15 human clinical studies demonstrated there is an association between hyperhomocysteinemia and cognitive impairment in persons with Parkinson's disease³⁸. Mini-mental status examination of orientation, registration, attention, recall, naming, repetition, syntax, reading, commands, and copying was used for evaluation³⁸.

The indirect methyl donor, Choline, and direct methyl donor, Betaine, also mitigate hypermethylation of DNA and proteins in accelerated brain aging³⁷. Choline and Betaine are essential nutrients that additionally fortify the neural membrane, enhance neuro-metabolism, and regulate neurotransmitter synthesis³⁷. Human clinical trials using Betaine or Choline supplementation demonstrate a significant reduction in plasma homocysteine concentrations³⁷.

Nootropic and Systemic Effects

Human studies demonstrate that Methylcobalamin has clinical benefit in the duration, cardinal symptoms, and severity of Parkinson's disease as well as mild cognitive impairment, adaptive behavior in autism spectrum disorder, late-life depression, and peripheral neuropathy^{38, 39, 40}. Choline and Betaine are essential in impaired folate-dependent methylation, especially in cases of folate deficiency, vitamin B12 deficiency, or alcoholism³⁷.

Methylcobalamin is effective in non-neurological conditions including congenital birth defects, dysfunction calcium metabolism, and diseases of the cardiovascular, renal, and gastrointestinal systems⁴¹. Meta-analysis of epidemiological studies demonstrate an inverse relationship between serum Choline and Betaine concentrations and the incidence of colorectal, breast, nasopharyngeal, hepatocellular, and pulmonary neoplasms⁴².

5-Methyltetrahydrofolate and Eucommia ulmoides Oliver in the Neuroplasticity Neurotransmitter Hypothesis

Modulation of Biological Brain Aging to Enhance Healthspan

The Neuroplasticity Neurotransmitter Hypothesis posits that the capacity for early neurodevelopment, post-injury neural recovery, and neural enhancement in adulthood, known as neuroplasticity, is modulated largely by neurotransmitters, the control of which is weakened in maladaptive brain aging^{43, 44, 45}. This is validated in clinical, pathological, and imaging studies in human and animal models of normal aging and neurodegenerative disease^{43, 44, 45}.

These neurotransmitters include acetylcholine, dopamine, serotonin, adrenaline, and norepinephrine that are critical for attention, learning, cognition, memory, impulse control, reward-motivation behavior, mood, and somatic manifestations of stress^{43, 44, 45}. Significant inter-person heterogeneity in baseline neurotransmitter reserves determine the capacity for neuronal recruitment, activation, and communication in hippocampal, frontal-temporal-parietal, subcortical, limbic, auditory, and visual neural networks as well as cerebral blood flow patterns and rate of grey matter atrophy^{43, 44, 45}.

The substance in BrainTheory™ N^{o} 12 that facilitates neuroplasticity is 5-Methyltetrahydrofolate. 5-Methyltetrahydrofolate is the bioactive and neuro-available form of folate that is critical in the production of neuromodulatory neurotransmitters⁴⁶. Evidence from a randomized, controlled human clinical trial demonstrated that bioactive folic acid supplementation in persons with mild cognitive impairment improves cognitive function, as measured by pre- and post-intervention full scale IQ, verbal IQ, verbal comprehension index, and working memory index, and deceases dementia-related biomarkers including beta-amyloid, homocysteine, S-adenosylmethionine, and amyloid precursor protein mRNA expression⁴⁶.

Phytochemicals in Eucommia ulmoides Oliver have neuroplasticity properties^{47, 48}. Laboratory models demonstrate that phytochemicals in E. ulmoides inhibit acetylcholinesterase, the enzyme that degrades acetylcholine, in lesioned brain regions including the frontal cortex and hippocampus⁴⁷. In the lesioned brain, via phytochemical modulation of different molecular pathways, mitochondrial structure and function is stabilized and neurotransmitter and brain-derived neurotrophic factor concentrations are increased⁴⁷. Phytochemicals in E. ulmoides also regulate the ubiquitin-proteasome system to increase neuron survival and control neuron apoptosis⁴⁸.

Nootropic and Systemic Effects

Human studies demonstrate that bioactive folate has clinical benefit in selective serotonin reuptake inhibitor (SSRI)-resistant major depression, analgesic effect in chronic pain syndromes, incidence of neural tube defects, and autism spectrum disorder as well as neuromodulatory properties in the primitive limbic system of persons with schizophrenia^{49, 50, 51}.

Laboratory models demonstrate that active phytochemicals in E. ulmoides significantly improve short-term and working memory impairment and partially reverse acquired learning deficits⁴⁸. Studies also demonstrate that phytochemicals in E. ulmoides have a calming effect via promotion of synapsin I, which increases serotonin release with resultant anti-fatigue and antidepressant effects⁴⁸. In epilepsy-associated memory impairment, phytochemicals in E. ulmoides protect memory-associated CA-2 hippocampal neuron degeneration⁴⁸.

E. ulmoides also has potent anti-aging effects via augmentation of collagen synthesis and UV radiation-induced photo-aging⁴⁸. Human and laboratory studies demonstrate non-neurological benefits of E. ulmoides in immunoprotection, hypertension, cardiovascular remodeling, hyperlipidemia, diabetes mellitus, erectile dysfunction, glaucoma, bone metabolism and diseases of the hepatic, renal, and pulmonary systems⁴⁸.

Trans-Pterostilbene in the Telomere Mortality Hypothesis

Modulation of Biological Brain Aging to Enhance Healthspan

In maladaptive brain aging, the production of abnormal neuronal substrates increases as a result of the erroneous integration, excision, and modification of genetic material originating from chromosomes^{52, 53, 54}. Chromosomes change in morphology and decrease in length over time via the degeneration of telomeres, which are structures at the distal ends of chromosomes purposed to preserve chromosomal integrity^{52, 53, 54}. The preservation of chromosomal integrity is due to telomerase, an enzyme produced in a subset of non-human animals to preserve telomeres and thereby maintain original chromosomal function^{52, 53, 54}.

The genotype and phenotype of a subset non-human animals such as those in the Nephropidae family are not significantly altered over a lifespan secondary to sufficient production of telomerase^{52, 53, 54}. The adult human has minimal telomerase reserves within highly sensitive, eloquent regions of the brain including the hippocampus^{52, 53, 54}.

The substance in BrainTheory™ N^{o} 12 that increases telomere length is Trans-Pterostilbene, the bioactive and neuro-available component of resveratrol. Trans-Pterostilbene restores RNA splicing ability and is associated with decreased human telomerase reverse transcriptase (hTERT) activity^{55,56}. RNA splicing involves post-production protein modification to excise and integrate target protein regions⁵⁵. Splicing errors have significant downstream effects that propagate deleterious changes in neurons and shortens telomeres⁵⁵. Trans-Pterostilbene also has the capacity to regulate apoptosis to maintain systemic homeostasis⁵⁵. The latter will be discussed in the ‘Dose-Dependent Substrates’ section.

Evidence from a randomized, double-blind, placebo-controlled human clinical trial demonstrated that Trans-Pterostilbene significantly improves function in and slows progression of neurodegenerative processes in amyotrophic lateral sclerosis (ALS)⁵⁵. Quality of life factors including speech, salivation, swallowing, handwriting, feeding, dressing, hygiene, walking, climbing stairs, and pulmonary function as well as muscular strength and skeletal muscle:fat weight ratio were evaluated⁵⁵. ALS an appropriate model neurodegenerative disease given that corticospinal motor neuron degeneration and neuromuscular unit atrophy are pathognomonic of of maladaptive brain aging.

Nootropic and Systemic Effects

Human studies demonstrate that Trans-Pterostilebene significantly improves mini-mental status cognitive examination and the ability to perform activities of daily living in persons with neurodegenerative disease^{57, 58}. Human studies demonstrate that Trans-Pterostilebene mimics anti-aging benefits of caloric restriction through the critical aging pathway SIRT1⁵⁷, a system involved in the onset of neurodegeneration. Trans-Pterostilebene demonstrates clinical benefit in neurological diseases such as ischemic stroke and non-neurological autoimmune diseases associated with its potent antioxidant, anti-inflammatory, and anti-immunosenescence properties⁵⁸. Pre-clinical studies also demonstrate that Trans-Pterostilebene facilitates hippocampal regeneration at a low to moderate dose by enhancing neurogenesis and inhibiting apoptosis of normal cells^{59, 60, 61}.

Cholecalciferol in the Immunosenescence Hypothesis

Modulation of Biological Brain Aging to Enhance Healthspan

The immunosenescence hypothesis posits that the immune system has innate and adaptive cell lines that become senescent, or senile, in maladaptive brain aging⁶². The innate immune response is non-specific and ubiquitously protects points of entry, namely the integumentary system and mucous membranes, whereas the adaptive immune response is targeted, specific, with inherent memory capabilities⁶². The adaptive immune system is comprised of antibody-generating B cells and helper or killer T cells⁶³. Helper T-cells trigger an immuno-inflammatory response and killer T-cells directly neutralize pathogens⁶³. In a functional immune system, this hyperdynamic response is limited in space and time⁶³.

In the hyperactive young, adaptive immune system, targets may be supra-specific and identify self-antigens as foreign, hence the propensity for autoimmune diseases in younger populations^{62, 63}. As the same young persons age, autoimmune diseases defervesce as a result of loss of this hyperactive self-antigen response^{62, 63}. In maladaptive brain aging, T-cells and molecular checkpoints become dysfunctional^{63, 64}. As a result, multiple aberrant phenomena may occur. Chronic, dormant infectious agents can reactivate and previously targeted immuno-inflammatory responses may become non-specific and unregulated (not limited in space and time)^{63, 64}. The dysfunctional aging immune system self-attacks as well and usually with a more fulminant course given there are less neurological reservese^{63, 64}.

The substance in BrainTheory™ N^{o} 12 that regulates immunosenescence is Cholecalciferol, the bioactive and neuro-available form of vitamin D⁶⁵. Evidence from a randomized, controlled, double-blind human clinical trial demonstrated that high doses of bioactive vitamin D are associated with fewer new brain lesions, reduced disability, and improved mobility when used as adjunctive therapy in multiple sclerosis⁶⁵.

However, doses greater than the recommended allowance should be reserved for hyperactive immune system responses in autoimmune diseases^{62, 63, 64}. In the aging immune system, a low to moderate dose of Cholecalciferol, to be described in the ‘Dose-Dependent Substances’ section, optimizes T-cell function and reduces neuron microglial response^{62, 63, 64}. Human and laboratory models demonstrate that bioactive vitamin D increases neuronal development, function, and survival via neurotrophins and promotes growth of axons and myelin^{66, 67}. This has implications in the pathophysiology of a range of processes from neurodegenerative to neurorestorative^{66, 67}.

Anti-inflammatory omega-3 (ω-3) polyunsaturated fatty acids (PUFAs) down-regulate immunosenescence^{66, 69}. ω-3 PUFAs balance existing T-cells subtypes, regulate T-cell production pathways, and effect the ability of antigen presenting cells to signal T-cells^{68, 69}. Laboratory models of immunosuppression demonstrate immune system enhancement with ω-3 PUFA supplementation^{68, 69}. Human studies additionally demonstrate positive effect on telomere length associated with enhanced healthspan⁷⁰, preservation of brain volume, and mitigation of amyloid proteinopathy⁷¹. A meta-analysis of seven randomized controlled trials investigating the effect of ω-3 PUFAs on mild cognitive impairment demonstrated that supplementation improves cognition in older adults with mild cognitive impairment⁷⁰.

Nootropic and Systemic Effects

Cholecalciferol deficiency plays a major role in a spectrum of neurological and psychiatric disorders, including painful peripheral nerve injury, depression, and epileptic seizures as well as medical conditions including rheumatoid arthritis, type 1 diabetes, lupus, and inflammatory bowel disease^{66, 72, 73}.

ω-3 PUFAs plays role in attention, processing speed, and recall in persons with mild cognitive impairment as well as neurological processes including early neurodevelopment, depression, and attention deficit hyperactivity disorder⁷¹. ω-3 PUFAs also demonstrate benefit in non-neurological conditions including cardiovascular disease, age-related macular degeneration, rheumatoid arthritis, inflammatory bowel disease, childhood allergies, and pulmonary function in cystic fibrosis⁷¹.

Apigenin in the Proteinopathy Hypothesis

Modulation of Biological Brain Aging to Enhance Healthspan

The Proteinopathy Hypothesis posits that, in maladaptive brain aging, protein misfolding and errors in protein post-processing result in the subsequent buildup of pathological protein compounds, known as proteinopathy^{74, 75}. For context, neurodegenerative diseases including amyotrophic lateral sclerosis (ALS), frontotemporal dementia (FTD), Alzheimer's disease (AD), cerebral amyloid angiopathy (CAA), Parkinson's disease (PD), and chronic traumatic encephalopathy (CTE) are primarily proteinopathies⁷⁵. The former two are characterized by the abnormal accumulation of TDP-43 protein⁷⁵. AD is characterized by the abnormal accumulation of tau and amyloid proteins⁷⁵. CAA is characterized by the abnormal accumulation of amyloid protein⁷⁵. PD is characterized by the abnormal accumulation of alpha-synuclein protein⁷⁵. CTE is characterized by the abnormal accumulation of both tau and TDP-43 proteins⁷⁵. Clinical presentations are based on the localization of pathological protein deposition. Clusters of differentiation, or CD, cells are critical in immune system signaling and protein expression⁷⁶. CD-40 regulates microglial immuno-inflammation in the nervous system and is dysfunctional in neurodegenerative disease such as ALS, AD, PD, prion diseases, and HIV-associated dementia as well as immuno-inflammatory diseases such as multiple sclerosis⁷⁶.

The substance in BrainTheory™ N^{o} 12 that mitigates proteinopathy is Apigenin. Apigenin is a flavonoid that prevents the buildup of misfolded amyloid protein, tau protein, alpha-synuclein protein⁷⁷ and modulates CD-40 expression⁷⁶. Evidence from critical appraisal of human studies demonstrates that Apigenin improves memory, learning, and sleep in both persons with Alzheimer's disease and healthy persons^{77, 78}. Human studies show that Apigenin promotes brain-derived neurotrophic factors to augment long term memory, promote neurogenesis, and support angiogenesis^{79, 80, 81}.

Nootropic and Systemic Effects

Apigenin is a non-addictive alternative to anxiolytic agents secondary to its GABA-nergic mimicry and antagonistic effects on stress-induced hypercortisolism⁷⁸. Apigenin has antidepressant and anti-insomnia properties as well as anti-carcinogenic and anti-viral properties^{78, 82}.

Luteolin in the Glymphatic Dysfunction Hypothesis

Modulation of Biological Brain Aging to Enhance Healthspan

The Glymphatic Dysfunction Hypothesis posits that the brain has a glial-dependent clearance system for waste and metabolic byproducts termed the glymphatic system, as the brain itself lacks true lymphatic vessels^{83, 84}. The glymphatic system transports waste and metabolic byproducts, such as those that buildup in proteinopathies, from the interstitial fluid^{83, 84}. Waste is then transferred to the true lymphatic system outside of the brain via the meningeal dura, cranial nerves, and blood vessels that exit the skull base. This occurs through the blood brain barrier, the permeability and integrity of which is largely regulated by mast cells⁸⁵. The glymphatic system is impaired in maladaptive brain aging as well as neurodegenerative diseases, traumatic brain injury, intra-cerebral hemorrhage, and ischemic stroke^{83, 84, 85}.

The substance in BrainTheory™ N^{o} 12 that facilitates neurotoxic waste product clearance is Luteolin. Luteolin is a flavonoid that regulates neuronal mast cells to facilitate the clearance of neurotoxic waste through the blood-brain barrier. Evidence from critical appraisal of human studies demonstrates that Luteolin improves ‘brain fog,’ cognition, concentration, and multitasking in the healthy brain⁸⁵.

Luteolin promotes the production and distribution of different neurotrophins that stimulate neuronal survival, development, and function⁸⁵. In both immunosenescence and the hyperactive immune system, Luteolin's potent anti-inflammatory properties have the capacity to alter transcription of microglial cells to a neuroprotective phenotype^{86, 87, 88}.

Nootropic and Systemic Effects

Human studies demonstrate that Luteolin improves attention and sociability in autism spectrum disorder⁸⁹ and is protective in fibromyalgia, fatigue syndromes, celiac disease, and other diseases associated with increased mast cells⁸⁵.

Melatonin in the Circadian Clock Epigenetics Hypothesis

Modulation of Biological Brain Aging to Enhance Healthspan

The Circadian Clock Hypothesis posits that the circadian clock becomes desynchronized in maladaptive brain aging and becomes dysfunctional in the pre-clinical stages of neurodegenerative diseases such as Alzheimer's⁹⁰. Circadian clocks are automated molecular feedback loops that coordinate time-based rhythms (circadian rhythms) of gene expression, neuronal function, neurophysiological processes, and behavior⁹⁰. The circadian system is preserved across species from insects to humans, and the aging circadian system is well-understood in organisms with accelerated life stages such a fruit flies⁹⁰. Mutations in clock genes, which are essential to facilitating automated molecular feedback loops, are associated with decreased lifespan and healthspan⁹⁰.

The substance in BrainTheory™ N^{o} 12 that resets the circadian clock is Melatonin. Melatonin is an endogenous substance produced in the brain's pineal gland and other specific brain regions⁹¹. Melatonin balances the production of clock proteins responsible for the brain's pituitary and hypothalamic functions dependent on automated time-based molecular feedback loops⁹¹. The pituitary gland and hypothalamus are responsible for neurological and non-neurological functions and behaviors, including but not limiting to muscle and bone growth, fat distribution, appetite, gastrointestinal motility, water retention, blood pressure, glycemic control, fertility, arousal, bonding, stress, metabolism, energy, temperature control, memory, and learning^{90, 91}. Evidence from human studies demonstrate that after a battery of neurocognitive testing, Melatonin supplementation in persons with mild cognitive impairment is associated with significant improvement in cognition⁹¹.

Melatonin has potent anti-aging properties as a result of caloric restriction mimicry via the SIRT-1 pathway and telomerase activation⁹². Evidence also demonstrates efficacy in mitigating cerebral ischemia, neurodegenerative diseases and proteinopathies, reductive oxidative stress, inflammation, and immunosenescence⁹³. Dose dependence will be discussed in the ‘Dose-Dependent Substrates’ section.

Nootropic and Systemic Effects

Human studies demonstrate that normalization of the circadian rhythm is critical in addiction treatment with application for Melatonin usage⁹⁴. Human studies also demonstrate Melatonin is effective as an adjunctive treatment for migraine because of its anti-CGRP properties, similar in mechanism of action to pharmaceutical anti-CGRP agents⁹⁵.

Magnesium L-Threonate in the Calcium-Dependent Synaptic Plasticity Hypothesis

Modulation of Biological Brain Aging to Enhance Healthspan

The Calcium-Dependent Synaptic Plasticity Hypothesis posits that dysregulation of neuronal molecular calcium homeostasis occurs in maladaptive brain aging via impairment of synaptic plasticity^{96, 97}. There are two main forms of synaptic plasticity, long-term potentiation (LTP) and long-term depression (LTD)^{96, 97}. LTP is critical in several afferent (pre-synaptic) pathways of the hippocampus, amygdala, visual, somatosensory, and prefrontal cortices^{96, 97}. LTP increases synaptic transmission and memory formation, and LTD decreases synaptic transmission and results memory loss^{96, 97}. In maladaptive brain aging, to achieve the same level of synaptic transmission in LTP, a higher induction threshold must be reached and is difficult to maintain^{96, 97}. Conversely, in maladaptive brain aging, the LTD induction threshold is lowered and easier to attain^{96, 97}. This low relative LTP/LTD ratio is associated with maladaptive age-related cognitive decline, deficits in memory and learning, and the ability to process, integrate, and consolidate new information^{96, 97}.

Unregulated post-synaptic calcium release is the result of dysfunctional neurotransmitter-associated communication, namely glutamate. This occurs at AMPA receptors, NMDA receptors, L-dependent voltage-gated calcium channels (VDCC), plasma membrane ATPase, and within intracellular endoplasmic reticulum and mitochondria^{96, 97}. Unregulated post-synaptic calcium release is also associated with aberrant phosphorylation, protease activation, and caspase-related apoptosis^{96, 97}. Elevated calcium levels markedly increase the LTP induction threshold and down-regulate neural-selective activity-dependent genes responsible for longterm memory formation such as brain-derived neurotrophic factor (BDNF)^{96, 97}.

The substance in BrainTheory™ N^{o} 12 that modulates calcium-dependent synaptic plasticity is Magnesium L-Threonate. Magnesium L-Threonate is a bioactive, neuro-available form of magnesium with superior blood-brain-barrier penetration. Magnesium L-Threonate regulates molecular NMDA-receptor check points to induce neuroplasticity of synapses via lowering the LTP threshold. Evidence from a randomized, controlled, double-blind human clinical trial demonstrated that Magnesium L-Threonate functions as a synapse density enhancer that improves learning and memory in mild cognitive impairment⁹⁸ and moderate dementia⁹⁹. Human studies demonstrate that Magnesium L-Threonate is neuroprotective in cerebral edema related to ischemic stroke, mitigates cerebral vasospasm, and has anti-epileptogenic properties¹⁰⁰.

Nootropic and Systemic Effects

Human studies demonstrate Magnesium L-Threonate is an adjunctive treatment in migraines and has therapeutic potential in depression and chronic pain¹⁰⁰.

Apple Pectin Prebiotic in the Gut-Brain Axis Hypothesis

Modulation of Biological Brain Aging to Enhance Healthspan

The Gut-Brain Axis Hypothesis, also known as the Second Genome Hypothesis, posits that the relationship between enteric, central, and peripheral nervous systems becomes asynchronous in maladaptive brain aging¹⁰¹. The gut-brain axis plays an integral role in critical phases of neurodevelopment (cognitive, emotional, and social neural networks) and neurodegenerative processes that are preceded by decreased microbial diversity and composition¹⁰¹.

The enteric nervous system (ENS) and microbiota engage in critical bidirectional communication with the central, autonomic, and peripheral nervous systems¹⁰¹. Functions of the ENS include modulation of small intestinal permeability to mitigate entrance of pro-inflammatory cytokines, production of short-chain fatty acids, and independent production of neurotransmitters including dopamine, serotonin, norepinephrine, and gamma-aminobutyric acid¹⁰¹. Functions of the hypothalamic-pituitary-adrenal axis, circadian rhythm, and sympathetic and parasympathetic nervous systems influence these processes¹⁰¹. Loss of symbiotic microbial diversity and the shift towards pathological microbiota is associated with loss of ENS function, cognitive deficits, and behavioral changes¹⁰¹.

The substance in BrainTheory™ N^{o} 12 that balances the microbiota is Apple Pectin Prebiotic^{102, 103}. Apple Pectin is a prebiotic that promotes the production of butyrate, a byproduct of symbiotic microbiota, preserves diversity of symbiotic microbiota, and prevents a shift towards pathological microbiota^{102, 103}. Prebiotics differ from probiotics in that prebiotics provide nutritional substrates, such as butyrate, to existing microbiota, allowing intrinsic epigenetic changes to be made^{102, 103}. Butyrate specifically plays a critical rule in neuroepigenetics¹⁰⁴. Evidence from critical appraisal of 14 human studies in the literature demonstrate that prebiotics have a significantly positive effect on cognition and mood including affect, verbal episodic memory, immediate recall, and recognition¹⁰².

Nootropic and Systemic Effects

Prebiotics have clinical benefits in sleep organization, stress response and cortisol levels, alcoholism, cancer^{103, 104}, autism spectrum disorder¹⁰⁵, depression¹⁰⁶, pain and fatigue syndromes, multiple sclerosis, inflammatory bowel disease, cardiovascular disease, and obesity¹⁰⁷. Human studies also show that prebiotics strengthen immunity via mucosal defense, stabilize bioactive foods and supplements, optimize vitamin biosynthesis, mitigate immunosenescence, and enhance bone density metabolism^{103, 104}.

Dose-Dependent Substances

Trans-Pterostilbene Dose-Dependent Efficacy

There is not a standard recommended or allowable dose of resveratrol or Trans-Pterostilbene.

At low doses (to be described), resveratrol has superior blood-brain-barrier penetration, promotes stability in reductive-oxidative stress, augments telomerase activity, and increases cell survival proteins⁶⁰. At high doses (to be described), resveratrol inhibits RNA and DNA synthesis, causes structural chromosome damage, and is pro-apoptotic in self and non-self cells, hence the rationale for its usage in adjunctive cancer treatment⁶⁰.

An upper limit dose of 2.5 mg/kg of resveratrol is shown to promote longevity pathways⁶⁰. Bioavailability of the resveratrol derivative, Trans-Pterostilbene, is 80 to 95% whereas the bioavailability of resveratrol is 20%¹⁰⁸. Based on an approximate bioavailability of 80% and a dose of 2 mg/kg in a 70 kg (154 lb) adult, BrainTheory™ N^{o} 12 is composed of 100 mg of Trans-Pterostilbene¹⁰⁸.

Cholecalciferol Dose-Dependent Efficacy

While calciferol deficiency (less than 20 ng/ml) is associated with maladaptive health and aging outcomes, serum calciferol levels above the higher range of normal (greater than 50 ng/ml)¹⁰⁹ are associated with accelerated aging, central thymus involution, and decreased naïve T cell production¹⁰⁹. For example, the age-suppressor gene, Klotho, is a model of aging. When over-expressed, Klotho increases healthspan¹¹⁰. When knocked out in animal studies, it results in accelerated aging, short lifespan, accelerated atherosclerosis, skin atrophy, osteoporosis, and high plasma calcitriol levels¹¹⁰. For cholecalciferol supplementation, the tolerable upper intake level, defined as the highest average daily intake of a nutrient that is likely to pose no risk of adverse health effects for nearly all persons in the general population, is 4000 IU^{109, 110}. This equates to 100 mcg or 500% of the recommended daily allowance^{109, 110}. Given the efficiency of oral absorption of cholecalciferol is approximately 50%, BrainTheory™ N^{o} 12 uses 50 mcg (2000 IU or 250% recommended daily allowance)¹¹¹.

Melatonin Dose-Dependent Efficacy

There is not a standard recommended or allowable dose of melatonin. Melatonin has a dose-dependent effect on basal pituitary and hypothalamic hormone release¹¹². A double-blind randomized crossover study demonstrated that neurohypophyseal hormone release is augmented by low dose melatonin 0.5 mg and inhibited by higher dose melatonin 5 mg. BrainTheory™ N^{o} 12 is composed of 0.5 mg of melatonin¹¹².

Dosing of Other Substances

(R)-Alpha Lipoic Acid

There is not a standard recommended or allowable dose of Alpha Lipoic Acid. A daily dose of 200 to 2400 mg/day of ALA is safe and well-tolerated¹¹³. Alpha Lipoic Acid is a racemic mixture where the R-enantiomer, which is bioactive and neuro-available, is present in equal portion to the non-bioactive S-enantiomer⁹. Based on this evidence, BrainTheory™ N^{o} 12 is composed of 100 mg of (R)-Alpha Lipoic Acid.

Crocetin

There is not a standard recommended or allowable dose of Crocetin. In a human pharmacokinetics study, persons given different doses of saffron extract (56 mg and 84 mg) had undetectable serum concentration of saffron isomers including crocin, safranal, and picrocrocin¹¹⁴. However, the Crocetin isomer was detected at a significant concentration with a bioavailability of 40% (22.4 mg and 33.6 mg, respectively)¹¹⁴. Similarly, a related study demonstrated a maximum Crocetin serum concentration was achieved after administration of 22.5 mg¹¹⁴. To account for at least 50% loss during trans-cellular intestinal and blood brain barrier passage¹¹⁵, BrainTheory™ N^{o} 12 is composed of 50 mg of Crocetin.

Curcumin

The Joint United Nations and World Health Organization Expert Committee on Food Additives (JECFA) and the European Food Safety Authority (EFSA) have set an allowable daily curcumin intake of 0-3 mg/kg¹¹⁶. Therefore, BrainTheory™ N^{o} 12 uses 100 mg of crocetin. For a 70 kg (154 lb) person, this equates to 1.5 mg/kg.

Methods to enhance oral bioavailability of Curcumin are under investigation, however, serum concentrations may be underestimated¹¹⁷. Curcumin undergoes gastrointestinal biotransformation to glucuronidated, sulfated, and methylated metabolites. This results in low levels of free bioactive Curcumin¹¹⁷. To simplify the quantification of serum concentration in research methodologies, samples are largely treated with β-glucuronidase to hydrolyze the bioactive Curcumin-glucuronide conjugate¹¹⁷. However, β-Glucuronidase incompletely hydrolyzes sulfate-containing conjugates that are also major metabolites¹¹⁷.

Of note, co-supplementation with piperine is suggested to significantly increase Curcumin bioavailablity¹¹⁸. This may be a result of piperine-associated glucuronidation inhibition, which results in more free bioactive curcumin as compared to bioactive conjugates¹¹⁹.

Methylcobalamin

The recommended daily allowance of cobalamin is 2.4 mcg¹²⁰. However, studies have suggested that 4 to 7 mcg of bioactive cobalamin (Methylcobalamin) in persons 18 to 50 with normal absorption is optimal to mitigate molecular aging pathways¹²⁰. Given insignificant evidence exists for cobalamin toxicity, a tolerable upper intake level does not exist¹²¹. Approximately 10 to 30% of adults over 50 years of age have cobalamin malabsorption and the presumed 50% bioavailability in the general population cannot be assumed¹²¹. To account for 10% bioavailability based on an intake of 5 mcg Methylcobalamin, BrainTheory™ N^{o} 12 is composed of 50 mcg of Methylcobalamin.

5-Methyltetrahydrofolate

The recommended daily allowance of folate is 400 mcg and based on erythrocyte folate, plasma homocysteine, and plasma folate concentrations¹²¹. BrainTheory™ N^{o} 12 uses 400 mcg dietary folate equivalents of 5-Methyltetrahydrofolate. Dietary folate equivalents adjust for 50 percent lower bioavailability of folate acquired from food compared with that of folic acid¹²¹.

Apigenin

There is not a standard recommended or allowable dose of Apigenin. An open-label clinical trial evaluated the effect of a combination therapy including Apigenin 100 mg in persons with Alzheimer's disease, Parkinson's disease, and multiple sclerosis¹²². A trend towards clinical stabilization and reduction of specific biomarkers including abnormal protein deposition was observed without adverse effects¹²². BrainTheory™ N^{o} 12 is composed of 100 mg of Apigenin.

Luteolin

There is not a standard recommended or allowable dose of Luteolin. A prospective, open-label trial evaluating the tolerability and efficacy of a combination therapy including Luteolin 100 mg in children with autism spectrum disorder⁸⁵ demonstrated no adverse effects. Data analysis suggested improvement in adaptive functioning and reduction in Aberrant Behavior Checklist subscale scores⁸⁵. BrainTheory™ N^{o} 12 is composed of 100 mg of Luteolin.

Magnesium L-Threonate

The recommended daily allowance of magnesium is 310 to 420 mg¹²³. The tolerable upper intake levels for supplemental magnesium in adults is 350 mg¹²³. Data from the National Health and Nutrition Examination showed that average magnesium intake from diet alone is approximately 268 mg in males and 234 mg in females¹²³. There is not a standard recommended or allowable dose specifically for Magnesium L-Threonate¹²³.

High doses of supplemental magnesium are associated with diarrhea, most commonly with magnesium carbonate, chloride, gluconate, and oxide¹²³. Studies have demonstrated that Threonate does accumulate in the periphery to the same extent as other forms of magnesium¹²⁴. Studies demonstrate that Threonate concentration is approximately 20 μM in plasma and 100 μM in cerebrospinal fluid (CSF)¹²⁴. In the brain, physiological extracellular magnesium concentration is 0.8 mM¹²⁴. In maladaptive brain aging and early neurodegeneration, extracellular magnesium concentration is less than 0.6 mM¹²⁴.

A study evaluating supplementation with L-Threonate 675 mg demonstrated a significant increase in CSF Threonate concentration by 54%, magnesium concentration by 15%, and synaptic density by 67% at 4 weeks¹²⁴. Delayed analysis was due to prior studies demonstrating that adequate Threonate concentration takes at least 2 weeks to attain¹²⁴. The relationship between extracellular magnesium and synapse density are bell-shaped¹²⁴. When extracellular magnesium is increased beyond 0.8 mM, intracellular magnesium and synapse density are decreased¹²⁴. In consideration of these findings, BrainTheory™ N^{o} 12 is composed of a conservative dosage of 11.7 mg of Magnesium L-Threonate.

Apple Pectin

There is not a standard recommended or allowable dose of Apple Pectin Prebiotic. Although Apple Pectin Prebiotic stimulates endogenous production of short chain fatty acids (SCFAs) including butyrate, dose-dependence and pharmacokinetics are not well-studied¹²⁵. As such, BrainTheory™ N^{o} 12 uses 50 mg of Apple Pectin, a conservative dosage based on current pectin-only containing supplements.

Product Manufacturing

BrainTheory N^{o} 12 is sourced and manufactured in the USA, contains USDA organic ingredients, and is produced in an FDA-registered and GMP-certified facility (NutraCap Labs, Norcross, Ga.).

What is claimed:

1. Twelve evidence-based hypotheses that form the theory of brain aging, herein referred to as Brain Theory, posit that maladaptive brain aging is mitigated by means of modulating specific processes at 12 different molecular interfaces.

2. The 12 substances and their alternatives (claims 9-20) that formulate Brain Theory's representative dietary supplement, herein referred to as BrainTheory™ N^{o} 12, modulate brain aging processes via one or more proposed mechanisms of brain aging. A dietary supplement formulation with said purpose is not previously described.

3. The 12 substances and their alternatives (claims 9-20) that formulate BrainTheory™ N^{o} 12 are the bioactive and neuro-available forms of their original compound.

4. The combination of the bioactive, neuro-available substances and their alternatives (claims 9-20) that formulate BrainTheory™ N^{o} 12 work in coaction and do not effect respective potency or tolerability.

5. The bioactive, neuro-available substances and their alternatives (claims 9-20) that formulate BrainTheory™ N^{o} 12 demonstrate cognitive enhancement properties analogous to the properties of a nootropic.

6. The scientific literature demonstrates the bioactive, neuro-available substances and their alternatives (claims 9-20) that formulate BrainTheory™ N^{o} 12 have adjunctive efficacious properties in neurological and non-neurological disorders.

7. The scientific literature demonstrates the bioactive, neuro-available substances and their alternatives (claims 9-20) that formulate BrainTheory™ N^{o} 12 engage in evidence-based nocturnal-specific neurorestorative processes.

8. The efficacy and intended purpose of specific bioactive, neuro-available substances in BrainTheory™ N^{o} 12 (claims 14, 15, and 18) are dose-dependent.

9. The Neurological Reserve Hypothesis posits that brain reserve and cognitive reserve decrease in brain aging. In agreement with claims 1-3, (R)-Alpha Lipoic Acid, the bioactive and neuro-available form of endogenously produced Alpha-Lipoic Acid, is a viable representative of the Neurological Reserve Hypothesis that mitigates brain aging via counteracting its proposed underlying mechanism. In agreement with claim 5, (R)-Alpha Lipoic Acid also has nootropic properties and scientific literature that demonstrates benefit in neurological and non-neurological disorders to be described. In the foreseeable future, other substances to be determined will be utilized in formulation as dictated by scientific evidence.

10. The Reductive-Oxidative Stress Hypothesis posits that neurons shift from a stable reduced state to an unstable oxidized state in brain aging. In agreement with claims 1-3, Crocetin, the bioactive and neuro-available form of saffron, is a viable representative of the Reductive-Oxidative Stress Hypothesis that mitigates brain aging via counteracting its proposed underlying mechanism. In agreement with claim 5, Crocetin also has nootropic properties and scientific literature that demonstrates benefit in neurological and non-neurological disorders to be described. In the foreseeable future, other substances to be determined will be utilized in formulation as dictated by scientific evidence.

11. The Caloric Restriction Anti-Inflammation Hypothesis posits that caloric restriction and mimickers of caloric restriction have a potent anti-inflammatory effect in brain aging. In agreement with claims 1-3, Curcumin, the bioactive and neuro-available form of turmeric, is a viable representative of the Caloric Restriction Anti-Inflammation Hypothesis that mitigates brain aging by counteracting its proposed underlying mechanism. In agreement with claim 5, Curcumin also has nootropic properties and scientific literature that demonstrates benefit in neurological and non-neurological disorders to be described. In the foreseeable future, other substances to be determined will be utilized in formulation as dictated by scientific evidence.

12. The Homocysteine Metabolism Hypothesis posits that elevated homocysteine levels in brain aging leads to dysfunctional methylation. In agreement with claims 1-3, Methylcobalamin, the bioactive and neuro-available form of vitamin B12, is a viable representative of the Homocysteine-Metabolism Hypothesis that mitigates brain aging via counteracting its proposed underlying mechanism. A next generation substance with a comparable mechanism of action to be utilized in formulation is Choline. In agreement with claim 5, Methylcobalamin and Choline also have nootropic properties and scientific literature that demonstrate benefit in neurological and non-neurological disorders to be described. In the foreseeable future, other substances to be determined will be utilized in formulation as dictated by scientific evidence.

13. The Neurotransmitter Neuroplasticity Hypothesis posits that neurotransmitter imbalance impedes neuroplasticity in brain aging. In agreement with claims 1-3, 5-Methyltetrahydrofolate, the bioactive and neuro-available form of folate, is a viable representative of the Neurotransmitter Neuroplasticity Hypothesis that mitigates brain aging via counteracting its proposed underlying mechanism. Next generation substances with comparable mechanisms of action to be utilized in formulation are phytochemicals present in Eucommia ulmoides Oliver. In agreement with claim 5, 5-Methyltetrahydrofolate and E. ulmoides also have nootropic properties and scientific literature that demonstrate benefit in neurological and non-neurological disorders to be described. In the foreseeable future, other substances to be determined will be utilized in formulation as dictated by scientific evidence.

14. The Telomere Mortality Hypothesis posits that paucity of telomerase in telomeres accelerates chromosomal damage in brain aging. In agreement with claims 1-3, Trans-Pterostilbene, the bioactive and neuro-available form of resveratrol, is a viable dose-dependent representative of the Telomere Mortality Hypothesis that mitigates brain aging via counteracting its proposed underlying mechanism. In agreement with claim 5, Trans-Pterostilbene also has nootropic properties and scientific literature that demonstrates benefit in neurological and non-neurological disorders to be described. In the foreseeable future, other substances to be determined will be utilized in formulation as dictated by scientific evidence.

15. The Immunosenescence Hypothesis posits that T-cell dysfunction in brain aging triggers systemic unregulated inflammation and breach of the blood-brain-barrier. In agreement with claims 1-3, Cholecalciferol, the bioactive and neuro-available form of vitamin D, is a dose-dependent viable representative of the Immunosenescence Hypothesis that mitigates brain aging via counteracting its proposed underlying mechanism. A next generation substance with a comparable mechanism of action to be utilized in formulation is an Omega-3-Fatty Acid Compound. In agreement with claim 5, Cholecalciferol and an Omega-3-Fatty Acid Compound also have nootropic properties and scientific literature that demonstrate benefit in neurological and non-neurological disorders to be described. In the foreseeable future, other substances to be determined will be utilized in formulation as dictated by scientific evidence.

16. The Proteinopathy Hypothesis posits that protein misfolding facilitates the accumulation of neurodegenerative proteins in brain aging. In agreement with claims 1-3, Apigenin, a bioactive and neuro-available flavonoid, is a viable representative of the Proteinopathy Hypothesis that mitigates brain aging via counteracting its proposed underlying mechanism. In agreement with claim 5, Apigenin also has nootropic properties and scientific literature that demonstrates benefit in neurological and non-neurological disorders to be described. In the foreseeable future, other substances to be determined will be utilized in formulation as dictated by scientific evidence.

17. The Glymphatic Dysfunction Hypothesis posits that clearance of neurotoxic waste products in the brain through the blood brain barrier is impaired in brain aging. In agreement with claims 1-3, Luteolin, a bioactive and neuro-available flavonoid, is a viable representative of the Glymphatic Dysfunction Hypothesis that mitigates brain aging via counteracting its proposed underlying mechanism. In agreement with claim 5, Luteolin also has nootropic properties and scientific literature that demonstrates benefit in neurological and non-neurological disorders to be described. In the foreseeable future, other substances to be determined will be utilized in formulation as dictated by scientific evidence.

18. The Circadian Clock Epigenetics Hypothesis posits clock genes that regulate feedback loops of critical automated biological processes become dysfunctional in brain aging. In agreement with claims 1-3, Melatonin is an endogenous and viable dose-dependent representative of the Circadian Clock Epigenetics Hypothesis that mitigates brain aging via counteracting its proposed underlying mechanism. In agreement with claim 5, Melatonin also has nootropic properties and scientific literature that demonstrates benefit in neurological and non-neurological disorders to be described. In the foreseeable future, other substances to be determined will be utilized in formulation as dictated by scientific evidence.

19. The Calcium-Dependent Synaptic Plasticity Hypothesis posits that the ability to control intra-neuronal calcium levels leads reduced synaptic plasticity in brain aging. In agreement with claims 1-3, Magnesium L-Threonate, the bioactive and neuro-available form of magnesium, is a viable representative of the Calcium-Dependent Synaptic Plasticity Hypothesis that mitigates brain aging via counteracting its proposed underlying mechanism. In agreement with claim 5, Magnesium L-Threonate also has nootropic properties and scientific literature that demonstrates benefit in neurological and non-neurological disorders to be described. In the foreseeable future, other substances to be determined will be utilized in formulation as dictated by scientific evidence.

20. The Gut-Brain-Axis Hypothesis posits that imbalance in microbiome diversity damages the gut's neural network in brain aging. In agreement with claims 1-3, Apple Pectin Prebiotic produces butyrate that functions as a viable representative of the Gut-Brain-Axis Hypothesis that mitigates brain aging via counteracting its proposed underlying mechanism. In agreement with claim 5, Apple Pectin Prebiotic also has nootropic properties and scientific literature that demonstrates benefit in neurological and non-neurological disorders to be described. In the foreseeable future, other substances to be determined will be utilized in formulation as dictated by scientific evidence.
